(19) United States
(12) Patent Application Publication
(10) Pub. No.: US 2022/0409240 A1
(43) Pub. Date: Dec. 29, 2022

(54) TUNNELER INSTRUMENT AND METHOD FOR PLACEMENT OF VASCULAR GRAFTS

(71) Applicant: Scanlan International, Inc., St. Paul, MN (US)

(72) Inventors: Robert E. Kohler, Lake Elmo, MN (US); John Mendyke, Eden Prairie, MN (US); Koua Vang, Maplewood, MN (US); Kelsey Sievert, Lakeland, MN (US); Matthew V. Leyden, St. Paul, MN (US); Charles W. Weber, St. Paul, MN (US)

(21) Appl. No.: 17/777,401

(22) PCT Filed: Nov. 18, 2020
(86) PCT No.: PCT/US2020/060997

§ 371 Date: May 17, 2022

Related U.S. Application Data
(60) Provisional application No. 62/936,839, filed on Nov. 18, 2019.

(51) Int. Cl. A61B 17/34

(52) U.S. Cl. CPC A61B 17/3468; A61B 2017/00473

(57) ABSTRACT

An instrument and method for placement of vascular grafts. Embodiments of the instrument comprise an elongated shaft, a clamp and a tip. The shaft includes a distal end portion, a proximal end portion, and optionally a handle at the proximal end portion. The clamp and/or the shaft are configured to removably couple the clamp to the distal end portion of the shaft. The tip and/or the instrument are configured to removably couple the tip to the instrument. In embodiments the tip covers the clamp.

### CROSS-REFERENCE TO RELATED APPLICATION

This application is a national phase application of PCT Application No. PCT/US2020/060997, internationally filed on Nov. 18, 2020, which claims the benefit of Provisional Application No. 62/936,839, filed Nov. 18, 2019, which are both incorporated herein by reference in their entireties for all purposes.

### FIELD

This disclosure relates generally to surgical instruments and methods. Embodiments include a tunneler instrument and method for placement of vascular grafts.

### BACKGROUND

Conduits such as vascular grafts are placed in patients for a variety of purposes. For example, vascular bypass grafts are used in the legs when the native flow is compromised due to atherosclerosis. Vascular access grafts are placed in arms for vascular access during dialysis. Vascular bypass and vascular access involve placing a new conduit in the subcutaneous space to provide a path for blood flow. The conduits may be synthetic or autologous vein material. The conduits may be placed by performing a tunneling procedure to create a channel under the skin for placement of the graft. The tunneling procedure may be performed using a tunneler instrument to create a channel. A secondary sheath may or may not be placed. The graft may be attached to the instrument and pulled back under the skin, and any sheath removed. Tunneler instruments and methods are disclosed, for example, in the Berry U.S. Pat. No. 5,306,240, the Herweck U.S. Pat. No. 6,565,594 and the Karwoski U.S. Pat. No. 7,628,795, and the Wortley U.S. Patent Application Publication 2004/0230204. There remains, however, a continuing need for improved graft placement instruments and methods.

### SUMMARY

Disclosed embodiments include an instrument and method for placing vascular grafts. An exemplary instrument comprises an elongated shaft having a distal end portion, a proximal end portion, and optionally a handle at the proximal end portion; a clamp, wherein the clamp and/or the shaft are configured to removably couple the clamp to the distal end portion of the shaft; and a tip, wherein the tip and/or the instrument are configured to removably couple the tip to the instrument.

Any or all of the examples may include a threaded joint to removably couple the tip to the instrument. In any or all examples, the tip is configured to be removably coupled to the clamp. Any or all examples may include a threaded joint to removably couple the tip to the clamp. In any or all examples the tip is configured to cover at least a portion of the clamp. In any or all examples the tip is configured to substantially cover portions of the clamp extending from the shaft.

In any or all of these examples the tip may include a tapered end portion. The tip may be a tubular member in any or all of these examples. In any or all of these examples the tip has an interior surface including threads; and the clamp has an exterior surface including threads, and wherein the threads of the tip are configured to be received by the threads of the clamp to removably couple the tip to the clamp.

In any or all of these examples the clamp comprises a bulldog clamp. A threaded joint may removably couple the clamp to the shaft in any or all of these examples. In examples, the clamp includes a threaded nut; and the shaft includes threads configured to receive the threaded nut.

An instrument in accordance with any or all of the above examples may further comprise interlock elements on one or both of the distal end portion of the shaft or the clamp to register the clamp in the shaft.

An instrument in accordance with any or all of the above examples may further comprise an elongated sheath having a distal end portion and a proximal end portion, wherein the sheath is configured to be slid and positioned over the shaft. In any or all of these examples the distal end portion of the sheath and/or the tip are configured to removably couple the tip to the sheath. Any or all of these examples may include a friction fit structure to removably couple the tip to the distal end portion of the sheath. A proximal end portion of the tip has a flange configured to be inserted into the distal end portion of the sheath in any or all of these examples.

The instrument in accordance with any or all of these examples may be part of a kit including a plurality of sheaths, including sheaths having different diameters. In any or all of these examples each sheath may be configured for one-time use.

The instrument in accordance with any or all of these examples may be part of a kit including a plurality of tips, including tips having different diameters. In any or all of these examples each tip may be configured for one-time use. The clamp may be configured for one-time use in any or all of these examples. In any or all of these examples the shaft, including any handle thereon, may be configured to be sterilized for multiple-time use.

### BRIEF DESCRIPTION OF THE DRAWINGS

FIG. 1 is an exploded view illustration of components of an instrument in accordance with embodiments.

FIG. 2 is an isometric illustration, partially in phantom, of a distal end portion of the instrument, in accordance with embodiments.

FIG. 3 is a cross sectional illustration of the distal end portion of the instrument, in accordance with embodiments.

FIG. 4 is a detailed isometric illustration of a distal end portion of a shaft of the instrument, in accordance with embodiments.

FIGS. 5 and 6 are isometric illustrations of a clamp of the instrument configured for mounting to the distal end portion of the shaft shown in FIG. 4, in accordance with embodiments.

FIG. 7 is an isometric illustration of a tip of the instrument, in accordance with embodiments.

FIG. 8 is a cross sectional illustration of the tip shown in FIG. 7, in accordance with embodiments.

FIG. 9 is a detailed isometric illustration of a distal end portion of a shaft of the instrument, in accordance with embodiments.

FIGS. 10 and 11 are illustrations of a clamp of the instrument configured for mounting to the distal end portion of the shaft shown in FIG. 9, in accordance with embodiments.

FIG. 12 is an isometric illustration of a tip of the instrument, in accordance with embodiments.

FIG. 13 is cross sectional illustration of the tip shown in FIG. 12, in accordance with embodiments.

FIG. 14 is an exploded view of the instrument, including the delivery component shown in FIG. 1 and the tip shown in FIGS. 12 and 13, in accordance with embodiments.

FIG. 15 is a detailed illustration of a portion of the instrument including the tip shown in FIG. 14 mounted to the distal end portion of the shaft.

### DETAILED DESCRIPTION

Instrument Structure

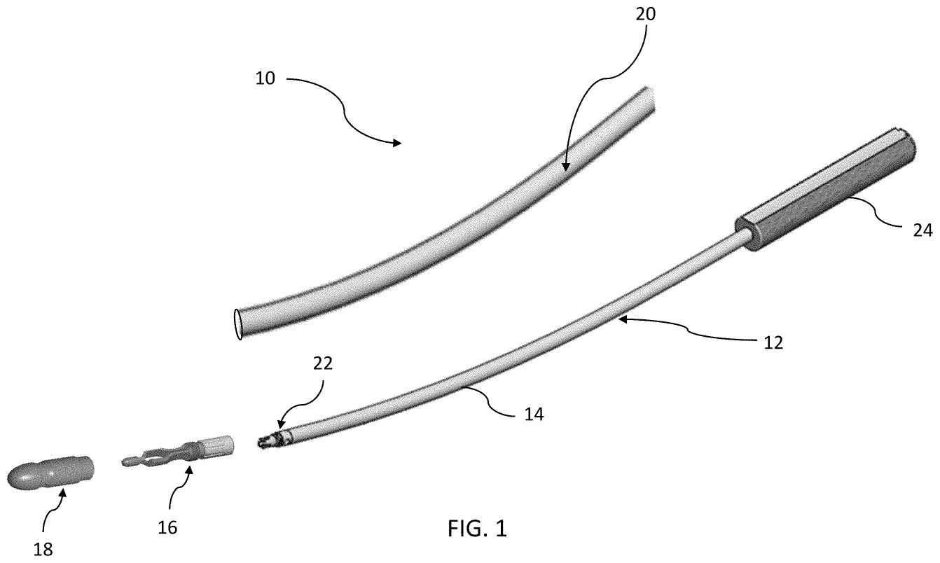

FIG. 1 illustrates components of an instrument in accordance with embodiments, such as tunneler 10. As shown, tunneler 10 includes a delivery component 12 having an elongated shaft 14, a clamp 16 and a tip 18. Embodiments may also include a sheath 20. Clamp 16 is removably attachable or couplable to a distal end portion 22 of the shaft 14. A handle 24 is located on a proximal end portion of the shaft 14 in the illustrated embodiments. Tip 18 is removably attachable or couplable to the instrument, such as to the clamp 16. As described below, for example, the tip 18 can cover all or portions of the clamp 16 and be removably coupled to the clamp. In embodiments including the sheath 20, the sheath is slid onto and positioned on the shaft 14. The sheath 20 mates with the tip 18 in embodiments. As described below, the tunneler 10 can be efficiently and effectively used in connection with patient procedures such as the placement of subcutaneous vascular grafts.

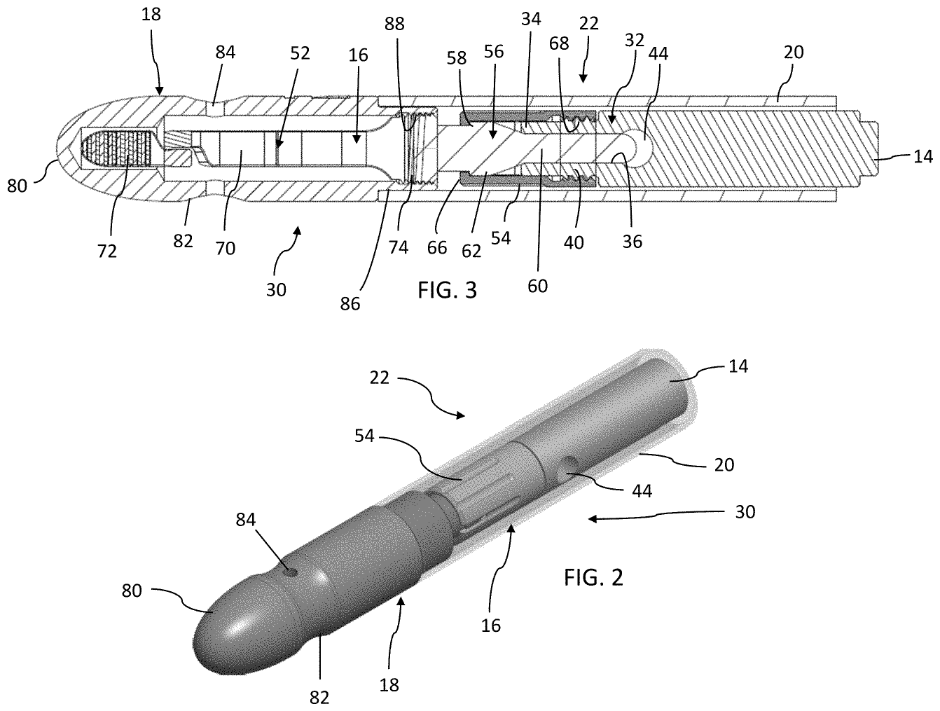

FIG. 2 illustrates the distal end portion 30 of embodiments of the tunneler 10, showing the sheath 20 (in phantom) over the shaft 14, with the clamp 16 coupled to the distal and portion 22 of the shaft, and the tip 18 coupled to the clamp 16. FIG. 3 is a sectional view of the distal end portion 30 of embodiments of tunneler 10 shown in FIG. 2.

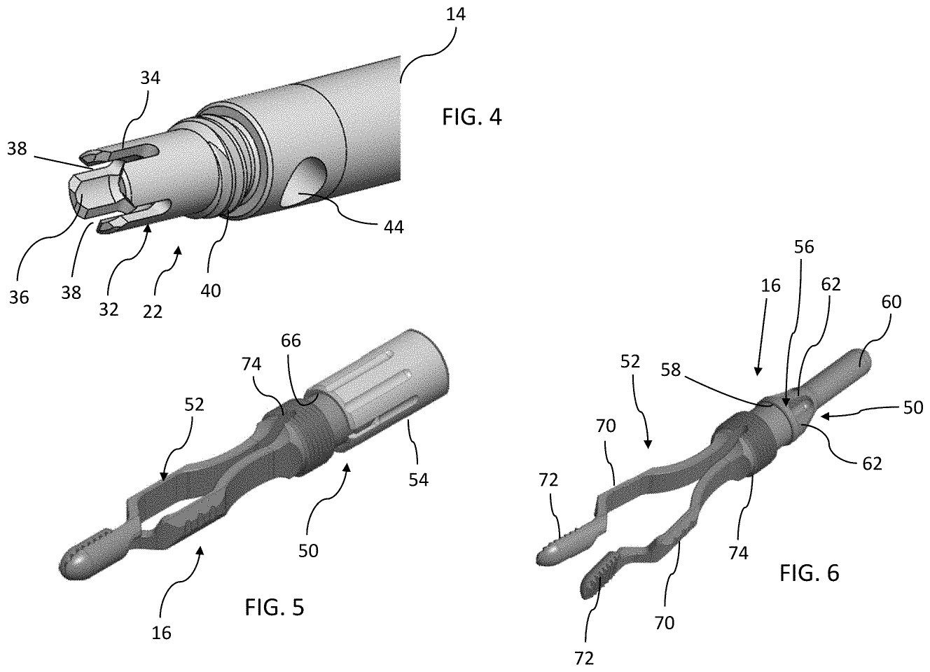

FIG. 4 illustrates the distal end portion 22 of shaft 14 in accordance with embodiments. As shown the shaft 14 includes a clamp mount 32 configured to receive the clamp 16. The illustrated embodiments of the clamp mount 32 include a cylindrical wall 34 having an opening 36 into its distal end and circumferentially spaced recesses 38, and threads 40 at the base of the cylindrical wall. An opening 44 extends through the distal end portion 22 of the shaft 14 in the illustrated embodiments (e.g., proximal of the threads 40). Opening 44 can, for example, be used in connection with cleaning of the shaft 14 (e.g., the opening 36).

FIGS. 5 and 6 illustrate embodiments of clamp 16. As shown, the clamp 16 includes a shaft mount 50, a clamp structure 52, and a nut 54 on the shaft mount. The illustrated shaft mount 50 includes a tapered base portion 56 having a shoulder 58 at its distal end portion, a pin 60 extending from its proximal end portion, and circumferentially spaced projections 62 extending from the tapered base portion. The shaft mount 50 is configured to mate with the clamp mount 32 on the shaft 14. As perhaps best shown in FIG. 3, the base portion 56, including the pin 60, are received in the opening 36 in the wall 34 of the clamp mount 32, with the projections 62 of the base portion fitting into the recesses 38 of the wall to register the clamp 16 to the shaft 14. Nut 54 is rotatably mounted to the shaft mount 50 and includes a lip 66 at its distal end portion and internal threads 68 at its proximal end portion. The nut 54 is screwed onto the distal end portion 22 of the shaft 14, with its threads 68 engaging the threads 40 on the shaft and its lip 66 engaging the shoulder 58 of the base portion 56, to releasably secure the shaft mount 50 and thereby the clamp 16 to the shaft. Elements of the shaft 14 and clamp 16 thereby cooperate and function as a threaded joint to removably join the shaft and clamp.

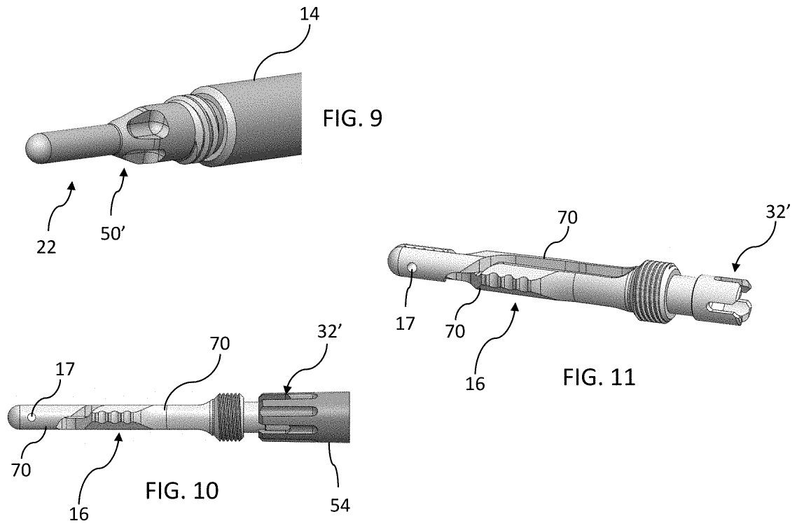

Other embodiments include other structures to removably couple the shaft 14 and clamp 16. By way of example, in embodiments such as those shown in FIGS. 9-11, a mount structure 50′ similar to that of the shaft mount 50 of the clamp 16 can be located on the distal end portion 22 of the shaft 14, and a mating mount structure 32′ similar to that of the clamp mount 32 of the shaft 14 can be located on the proximal end of the clamp 16 (i.e., the location of the clamp structures can be reversed from the locations of the embodiments illustrated in FIGS. 1-7). The clamp 16 illustrated in FIGS. 10 and 11 includes a through hole 17 through distal end portions of the spring arms 70. Other embodiments of clamp 16 (not shown) include one or more through holes such as 17 through only one of the spring arms 70 and/or at other locations on the clamp 16.

Clamp structure 52 is a bulldog clamp in the illustrated embodiments, and includes a pair of spring arms 70 having clamp surfaces 72 at their distal end portions. In the illustrated embodiments the spring arms 70 are configured to bias the clamp surfaces 72 to a closed or clamping position. As shown in FIGS. 3, 5 and 6, embodiments of the clamp 16 include threads 74. In the illustrated embodiments the threads 74 are located at the base of the clamp structure 52 and distal of the nut 54. Other embodiments include other clamp structures. For example, although not shown in FIGS. 5 and 6, the clamp 16 of the embodiments shown in these FIGs. may include one or more through holes such as through hole 17 described above in connection with the embodiments illustrated in FIGS. 10 and 11.

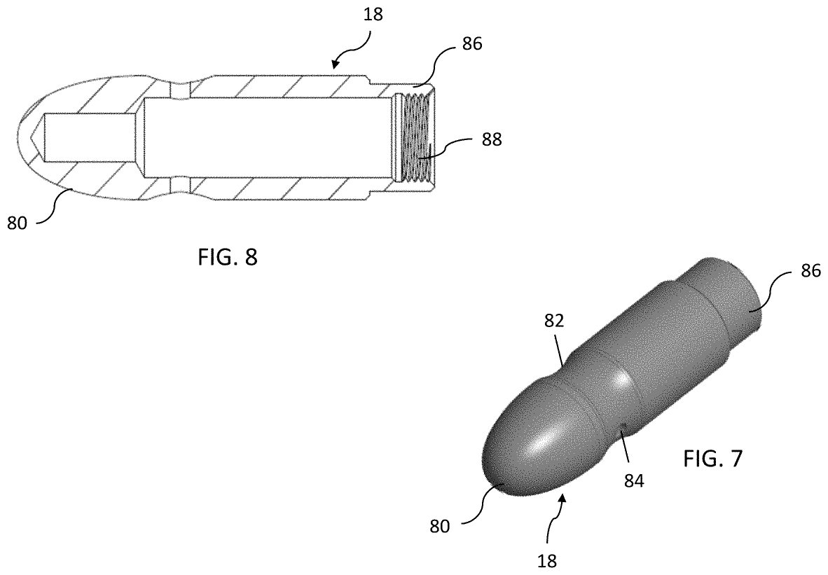

FIGS. 3, 7 and 8 illustrate embodiments of the tip 18. FIG. 8 is a sectional view of the tip 18 shown in FIG. 7. As shown, tip 18 is a generally tubular structure having a blunt or bullet-shaped distal end 80. The distal end 80 is closed in the illustrated embodiments. The illustrated embodiments include a reduced-diameter waist 82 having an opening 84 extending therethrough proximal of the distal end 80, and a reduced-diameter flange 86 at its proximal end portion. Threads 88 are located on an interior surface of the tip 18, and are shown on the flange 86 in the illustrated embodiments. When mounted to the instrument 10, tip 18 covers at least portions of the clamp 16. In the illustrated embodiments, tip 18 covers substantial portions of the clamp 16, such as the clamp structure 52. The tip 18 is screwed onto the distal portion of the clamp 16, with its threads 88 engaging the threads 74 on the clamp to releasably secure the tip to the clamp and thereby the instrument 10. Elements of the tip 18 and clamp 16 thereby cooperate and function as a threaded joint to removably join the tip and clamp. Other embodiments include other structures to removably couple the tip 18 and clamp 16.

FIGS. 2 and 3 illustrate the engagement of the tip 18 and sheath 20 in embodiments that include the sheath. As shown, the distal end portion of the sheath 20 extends over the flange 86 of the sheath. In the illustrated embodiment the outer diameter of the tip 18 and the outer diameter of the sheath 20 are the same at the location that these components join. The interior surface of the sheath 20 and exterior surface of the flange 86 of the tip 18 can be configured (e.g., the diameters of the components sized) to provide a friction fit between the components in embodiments.

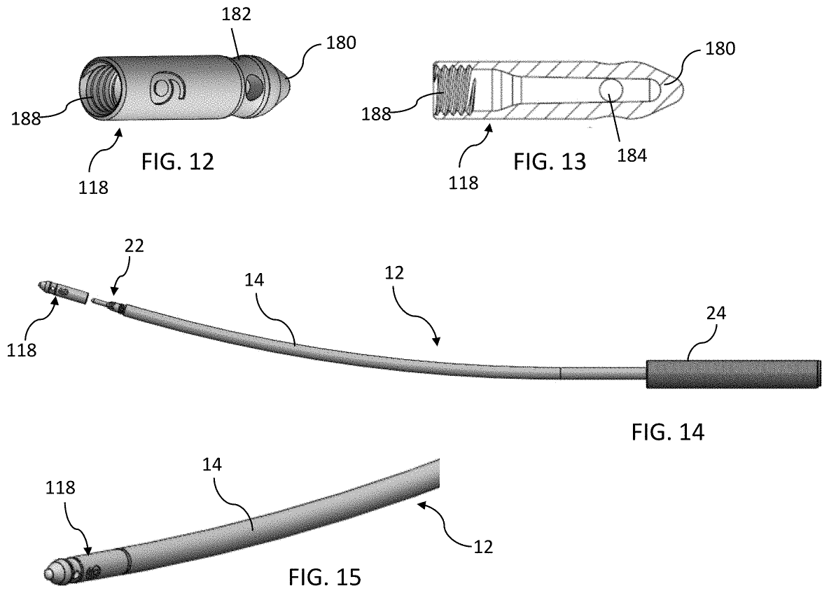

FIGS. 12 and 13 illustrate embodiments of a tip 118. FIG. 13 is a sectional view of the tip 118 shown in FIG. 12. As shown, tip 118 is a generally tubular structure having a blunt or bullet-shaped distal end 180, a waist 182 and an opening or aperture 184. The distal end 180 is closed in the illustrated embodiments. Unlike the tip 18 described above (e.g., in connection with FIGS. 7 and 8), tip 118 does not include a reduced-diameter waist. Threads 188 are located on an interior surface of a proximal end portion of the tip 118. In embodiments, tip 118 may have a smaller outer diameter than tips such as 18. In embodiments, for example, tip 118 has a diameter of 6 mm. As shown for example in FIGS. 14 and 15, tip 118 is configured to be mounted to the distal end portion 22 of the shaft 14. In the embodiments illustrated in FIGS. 14 and 15, the tip 118 and shaft 14 have the same outer diameters, and the proximal end of the tip 118 mates with a shoulder on the distal end portion 22 of the shaft to provide a generally continuous outer surface between the shaft and the waist 182 on distal end 180.

Method of Use

Instruments such as tunneler 10 may be used to form a passageway, or tunnel, through tissue of a patient. In embodiments, tunneling procedures can be performed to form subcutaneous tunnels, such as for example in the arms or legs of patients. Conduits or other devices, such as for example vascular bypass grafts or vascular access grafts, can be placed in the tunnels. In embodiments, instruments such as tunneler 10 can be used to place devices such as grafts in the tunnels formed by the tunneler.

Tunneler 10 can be set up and assembled, for example, by selecting and coupling a clamp 16 to the shaft 14, selecting and mounting a sheath 20 to the shaft (e.g., by sliding the sheath over the end of the clamp) and selecting and mounting a tip 18 to the clamp. A clinician then urges or forces the assembled tunneler 10 through the patient's skin at an entrance site and through subcutaneous tissue at a tunnel site, for example by manipulating the instrument while holding the handle 24. The tip 18 may exit the tissue through the patient's skin at an exit site. By this action the sheath is placed in the tissue tunnel. The clinician may then remove the tip 18 from the tunneler 10 and attach an end portion of the device to be implanted, such as a graft for example, to the distal end portion 30 of the tunneler. In embodiments, for example, the clinician can actuate the clamp structure 52 to clamp the graft to the tunneler 10. Alternatively or in addition, a clinician can tie the graft to the clamp 16, including for example extending the graft through the one or more through holes 17 described in connection with FIGS. 10 and 11. After the graft is attached to the tunneler 10, the shaft 14 is withdrawn from the patient through the sheath 20 while the sheath remains in the patient and tunnel (e.g., by manipulating the handle 24). By withdrawing the shaft 14 in this manner, the graft is pulled through the tunnel, inside the sheath 20. The end of the graft is then detached from the distal end portion 22 of the tunneler 10 (e.g., by releasing the clamp structure 52 and/or untying the graft). With the graft placed in the tunnel, the sheath 20 can then be pulled and removed from the patient while the graft remains in the tunnel. The clinician may, for example hold the end of the graft opposite the entrance or exit site from which the sheath 20 withdrawn. The graft is thereby placed in the tunnel.

In other embodiments using the tunneler 10 configured in the manner above, the graft can be attached to the tip 18, for example by tying the graft to the tip at the waist 82 and/or though the opening 84. The graft can then be placed into the tunnel by pulling the tunneler 10, including the sheath 20 and tip 18, through the tunnel in the patient's tissue. By this embodiment the tip 18 is not removed from the tunneler 10 and the shaft 14 is not withdrawn through the sheath 20 during placement of the graft.

In other embodiments the tunneler 10 is used without sheath 20 to place grafts. During graft placement procedures of these embodiments, the clamp 16 and tip 18 are mounted to the shaft 14 in a manners that can be substantially the same as or similar to those described above, but without the sheath 20, and the assembled tunneler 10 operated to create the tunnel in the patent's tissue in manners substantially the same as or similar to those described above. The graft may then be attached to the distal end portion of the tunneler 10 in manners that can be substantially the same as or similar to those described above, and tunneler withdrawn from the tunnel to place the graft in the tunnel in the patient's tissue. In these embodiments, for example, the graft can be attached to the clamp structure 52 (e.g., if the tip is removed) or to the tip (e.g., to the waist 82 and/or opening 84).

In yet other embodiments the tunneler 10 is used without the clamp 16. As shown for example in FIGS. 14 and 15, the tip 118 is mounted to the distal end portion 22 of the shaft 14. During use of embodiments of these types the graft can be attached to the tip 118, for example by tying the graft to the tip at the waist 182 and/or though the opening 184.

In embodiments, the shaft 14 and/or handle 24 are configured to be cleaned, sterilized and reused following placement procedures of the types described above. Shaft 14 and/or handle 24 can be polymer and/or metal devices in embodiments. In embodiments, the clamp 16, tip 18, 118 and/or sheath 20 are configured for one time use and disposal following the placement procedures. Clamp 16, tip 18, 118 and/or sheath 20 are polymer and/or metal devices in embodiments. In other embodiments the components of tunneler 10 are configured for other reuse and disposal options.

Packaging

One or more of the delivery component 12, clamp 16, tip 18,118 and sheath 20 can be packaged together for distribution. In embodiments, for example, a plurality of tips 18, 118 and/or a plurality of sheaths 20 having different diameters (e.g., 7 mm, 8 mm, 9 mm and/or 10 mm) can be packaged together for distribution (e.g., with or without the delivery component 12). The clinician can select the tip 18,118 and/or sheath 20 of a size appropriate for the given procedure, and assemble the tunneler 10 with the selected components. For example, the size of the tip 18, 118 and/or sheath 20 selected may depend on factors such as the nature of the tissue in which the tunnel is being formed, and/or the size (e.g. length, diameter) of the device (e.g., graft or other conduit) being implanted. In embodiments such as those described above, the tips 18, 118 and sheaths 20 may be packaged with a plurality of clamps 16. One or more delivery components 12 may also be included in the packaged combination.

Advantages

Instruments such as tunneler 10 and the associated methods described herein offer important advantages. For example, they offer clinicians the flexibility to select components that can be assembled into a tunneler 10 optimized for the circumstances presented for a given procedure. The tunneler 10 can be used in number of different ways (e.g., with or without a sheath 20; multiple ways to attach a graft to a distal end of the instrument), providing the clinician with the opportunity to perform procedures optimized to the circumstances presented and/or using personal preferences. The tunneler can be efficiently used to efficaciously perform tunneling procedures.

Although described with reference to embodiments, those of skill in the art will recognize that changes can be made in form and detail without departing from the spirit and scope of the claims.

1. An instrument, comprising:
an elongated shaft having a distal end portion, a proximal end portion, and optionally a handle at the proximal end portion;
a clamp, wherein the clamp and/or the shaft are configured to removably couple the clamp to the distal end portion of the shaft; and
a tip, wherein the tip and/or the instrument are configured to removably couple the tip to the instrument.

2. The instrument of claim 1 and including a threaded joint to removably couple the tip to the instrument.

3. The instrument of claim 1 wherein the tip is configured to be removably coupled to the clamp.

4. The instrument of claim 3 and including a threaded joint to removably couple the tip to the clamp.

5. The instrument of claim 1 wherein the tip is configured to cover at least a portion of the clamp.

6. The instrument of claim 1 wherein the tip is configured to substantially cover portions of the clamp extending from the shaft.

7. The instrument of claim 1 wherein the tip includes a tapered end portion.

8. The instrument of any of claim 1 wherein the tip is a tubular member.

9. The instrument of any of claim 1 wherein:
the tip has an interior surface including threads; and
the clamp has an exterior surface including threads, and wherein the threads of the tip are configured to be received by the threads of the clamp to removably couple the tip to the clamp.

10. The instrument of claim 1 wherein the clamp comprises a bulldog clamp.

11. The instrument of claim 1 and including a threaded joint to removably couple the clamp to the shaft.

12. The instrument of claim 1 wherein:
the clamp includes a threaded nut; and
the shaft includes threads configured to receive the threaded nut.

13. The instrument of claim 1 and further including interlock elements on one or both of the distal end portion of the shaft or the clamp to register the clamp in the shaft.

14. The instrument of claim 1 and further including an elongated sheath having a distal end portion and a proximal end portion, wherein the sheath is configured to be slid and positioned over the shaft.

15. The instrument of claim 14, wherein the distal end portion of the sheath and/or the tip are configured to removably couple the tip to the sheath.

16. The instrument of claim 15 and including friction fit structure to removably couple the tip to the distal end portion of the sheath.

17. The instrument of claim 14 wherein a proximal end portion of the tip has a flange configured to be inserted into the distal end portion of the sheath.

18. The instrument of claim 14 wherein the instrument is part of a kit including a plurality of sheaths, including sheaths having different diameters.

19. The instrument claim 18 wherein each sheath is configured for one-time use.

20. The instrument of claim 1 wherein the instrument is part of a kit including a plurality of tips, including tips having different diameters.

21. The instrument of claim 20 wherein each tip is configured for one-time use.

22. The instrument of claim 1 wherein the clamp is configured for one-time use.

23. The instrument of claim 1 wherein the shaft is configured to be sterilized for multiple-time use.
